(19) United States
(12) Patent Application Publication
(10) Pub. No.: US 2015/0366524 A1
(43) Pub. Date: Dec. 24, 2015

(54) RADIOGRAPHIC APPARATUS AND RADIOGRAPHIC SYSTEM

(71) Applicant: CANON KABUSHIKI KAISHA, Tokyo (JP)

(72) Inventors: Masataka Suzuki, Tokyo (JP); Kensuke Kobayashi, Tokyo (JP); Motoki Tagawa, Chigasaki-shi (JP); Katsushi Kato, Kawasaki-shi (JP)

(21) Appl. No.: 14/734,312

(22) Filed: Jun. 9, 2015

(30) Foreign Application Priority Data
Jun. 18, 2014 (JP) 2014-125731
Mar. 24, 2015 (JP) 2015-061684

(51) Int. Cl. A61B 6/00

(52) U.S. Cl. CPC A61B 6/4283

(57) ABSTRACT

A radiographic apparatus includes a radiation sensor panel and a housing that encloses the panel. The radiation sensor panel has a detection surface on which a converting element that detects radiation or light is disposed. The housing includes an incident, a slope, and a flat surface portion. Radiation enters the radiographic apparatus through the incident portion, which is located adjacent to the detection surface. The slope portion is located at a housing end portion and on a radiation sensor panel side opposite to the detection surface. The slope portion is inclined with respect to a direction of a housing thickness. The flat surface portion is located on the side of the radiation sensor panel opposite to the detection surface and is substantially parallel to a flat portion of the incident portion. The slope portion has an average thickness that is greater than an average thickness of the flat surface portion.

### BACKGROUND OF THE INVENTION

1. Field of the Invention

The present invention relates to radiographic apparatuses and radiographic systems.

2. Description of the Related Art

Radiographic apparatuses, which detect the distribution of the intensity of radiation that has penetrated an object and obtain radiation images of the object, have been widely used in the fields of industrial nondestructive inspections and medical diagnoses. Radiographic apparatuses are required to be strong enough to bear an impact resulting from, for example, unintended falling during use or an external force that can occur during radiographing. Radiographic apparatuses are also required to have a structure that is highly operable for easy handling or that loads fewer burdens on test subjects at the placement of the radiographic apparatuses.

Japanese Patent Laid-Open No. 2011-221361 discloses a radiographic apparatus in which a housing, which encloses a radiation sensor panel, has slope portions at its end portions. This structure facilitates raising of the radiographic apparatus, whereby the radiographic apparatus is easily inserted into a lower portion of a test subject during radiographing.

An impact resulting from falling or the like or an external force that occurs during radiographing is likely to be exerted on side walls of the housing of a radiographic apparatus. In the structure of the housing disclosed in Japanese Patent Laid-Open No. 2011-221361 having slope portions at its end portions, an impact or an external force is likely to be exerted on or around the slope portions besides the side walls of the housing. In such a case, stress concentration is likely to occur at or around the slope portions, whereby bending at or around the slope portions or buckling of the slope portions may occur.

### SUMMARY OF THE INVENTION

An aspect of the present invention is a radiographic apparatus having a housing that maintains its strength while the operability of the radiographic apparatus is retained.

According to an aspect of the present invention, a radiographic apparatus includes a radiation sensor panel having a detection surface on which a converting element that detects radiation or light is disposed, and a housing that encloses the radiation sensor panel, wherein the housing includes an incident portion through which the radiation enters the radiographic apparatus, wherein the incident portion is located adjacent to the detection surface of the radiation sensor panel, a slope portion that is located at an end portion of the housing and on a side of the radiation sensor panel opposite to the detection surface, wherein the slope portion is inclined with respect to a direction of a thickness of the housing, and a flat surface portion that is located on the side of the radiation sensor panel opposite to the detection surface and that is substantially parallel to a flat portion of the incident portion, and wherein the slope portion has an average thickness that is greater than an average thickness of the flat surface portion.

Further features of the present invention will become apparent from the following description of exemplary embodiments (with reference to the attached drawings).

### BRIEF DESCRIPTION OF THE DRAWINGS

FIG. 1A is a perspective view of a radiographic apparatus according to a first embodiment and FIG. 1B is a cross-sectional view of the radiographic apparatus.

FIG. 2 is a cross-sectional view of a housing of the radiographic apparatus according to the first embodiment.

FIG. 3 is a cross-sectional view of the housing of the radiographic apparatus according to the first embodiment.

FIG. 4 is a cross-sectional view of a radiographic apparatus according to a second embodiment.

FIGS. 5A and 5B are perspective views of a radiographic apparatus according to a third embodiment and FIG. 5C is a cross-sectional view of the radiographic apparatus.

FIG. 6A is a perspective view of a radiographic apparatus according to a fourth embodiment and FIGS. 6B and 6C are cross-sectional views of the radiographic apparatus.

FIG. 7 illustrates a radiographic system, which is an application example of the radiographic apparatus according to any of the first to fourth embodiments.

### DESCRIPTION OF THE EMBODIMENTS

### First Embodiment

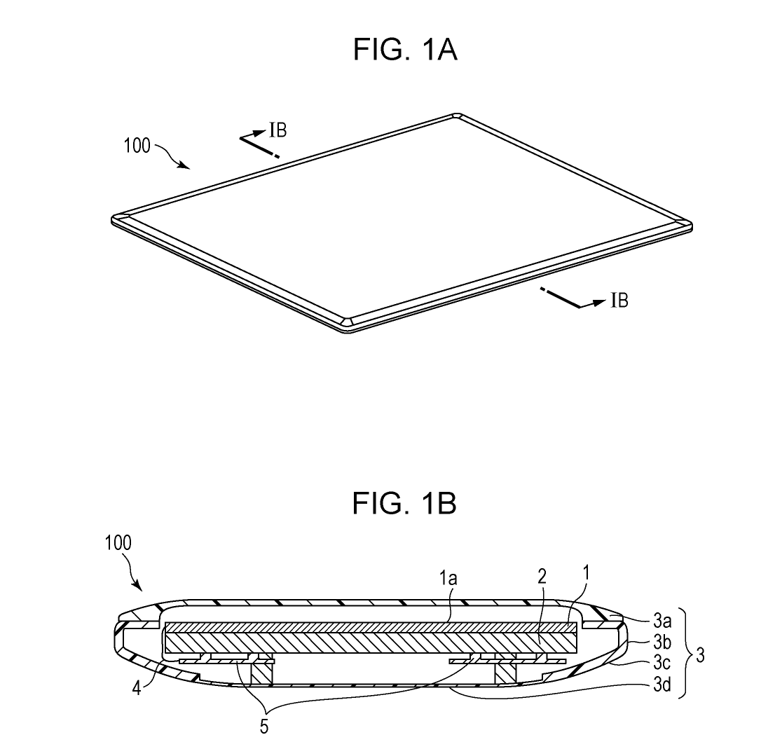

Referring to FIGS. 1A and 1B, a radiographic apparatus according to a first embodiment is described. FIG. 1A is a perspective view of a radiographic apparatus 100 according to a first embodiment. FIG. 1B is a cross-sectional view of the radiographic apparatus 100 according to the first embodiment taken along the line IB-IB.

The radiographic apparatus 100 includes at least a radiation sensor panel 1 and a housing 3.

The housing 3 encloses the radiation sensor panel 1. The housing 3 includes an incident portion 3a, a side surface portion 3b, a slope portion 3c, and a flat surface portion 3d. The radiographic apparatus 100 also includes a base 2, a flexible circuit board 4, and control boards 5.

Components of the radiographic apparatus 100 are described in detail below.

The radiation sensor panel 1 has a function of converting incident radiation into image signals. The radiation sensor panel 1 has a detection surface 1a on which converting elements, which detect radiation or light, are disposed. A fluorescent substance (not illustrated), which converts radiation into visible light, is disposed on the detection surface 1a. In this embodiment, MIS or PIN photoelectric converting elements that can detect visible light are used as examples of the converting elements. The radiation applied to the radiographic apparatus 100 causes the fluorescent substance to emit light, which is then converted into image signals by the photoelectric converting elements on the radiation sensor panel 1. Instead of the fluorescent substance and the photoelectric converting elements, the radiation sensor panel 1 may support converting elements of a direct conversion type that directly converts radiation into electric charges.

The control boards 5 have a function of controlling the radiation sensor panel 1. The control boards 5 are electrically connected to the radiation sensor panel 1 using flexible circuit boards 4. Various integrated circuits are provided on the flexible circuit boards 4 and the control boards 5. The integrated circuits include a driving circuit for driving the converting elements, a reading circuit for reading electric signals, and a control circuit for controlling at least one of the driving circuit and the reading circuit.

The housing 3 is described now. The housing 3 encloses the radiation sensor panel 1. As illustrated in FIG. 1B, the housing 3 includes an incident portion 3a, a side surface portion 3b, a slope portion 3c, and a flat surface portion 3d. The incident portion 3a is detachable from other components (or the body, below). The incident portion 3a is located adjacent to the detection surface 1a of the radiation sensor panel 1. The incident portion 3a has a flat portion, which is a surface that allows radiation to penetrate therethrough. Desirably, the flat portion of the incident portion 3a has a high radiation permeability to allow radiation to penetrate therethrough. The incident portion 3a is desirably light in weight and has a predetermined strength against impacts. Examples of the material of the incident portion 3a include resin and carbon fiber reinforced plastics (CFRP). The side surface portion 3b is located at the outer edge of the radiation sensor panel 1. The slope portion 3c and the flat surface portion 3d are located on the side of the radiation sensor panel 1 opposite to the detection surface 1a. The slope portion 3c is bent at the end portions of the housing 3 and inclined with respect to the thickness direction. The flat surface portion 3d has a surface substantially parallel to the incident portion 3a. Here, being substantially parallel is not limited to the case of being kept parallel in a strict sense. For example, being substantially parallel includes the structure in which surfaces are kept substantially parallel to each other although they are not parallel to each other in a strict sense due to an assembly error or time change. A substantially parallel flat surface portion represents a surface having the largest area within the same surface in the case where the surface has multiple flat portions. The average thickness of the slope portion 3c is greater than the average thickness of the flat surface portion 3d. The average thickness of the side surface portion 3b is greater than the average thickness of the flat surface portion 3d. The body of the housing 3 includes the side surface portion 3b, the slope portion 3c, and the flat surface portion 3d, which are integrated into one unit. The body having an integrated structure enhances the rigidity of the housing and facilitates manufacture (forming). Desirably, the body is strong enough to bear falling, an impact, or the like, light in weight for easy transportation, and highly operable. The body is made of a material such as magnesium, aluminum, CFRP, or fiber-reinforced resin. The load capacity of the incident portion 3a of the housing 3 is desirably 150 kg or greater. The load capacity at a local point having a diameter of 40 mm or smaller is desirably 100 kg or greater.

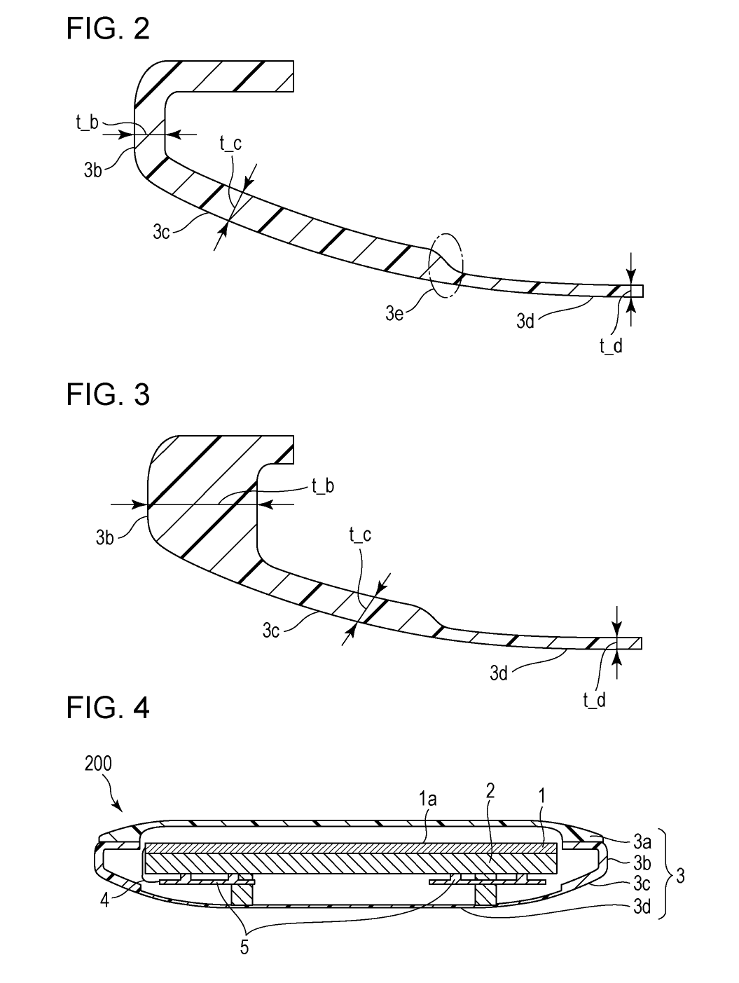

As illustrated in FIG. 2, in the housing 3, at least part of the slope portion 3c has a thickness that is the same as the average thickness of the flat surface portion 3d. This structure can prevent an increase of the weight of the housing 3 while the housing 3 retains a predetermined strength, unlike in the case where the entirety of the slope portion 3c has a thickness greater than the average thickness of the flat surface portion 3d. The difference in thickness between the slope portion 3c and the flat surface portion 3d gradually decreases in the housing 3. This structure can prevent stress concentration on a portion between the slope portion 3c and the flat surface portion 3d and prevent an increase in weight. Particularly, the flat surface portion 3d of the housing 3 has a greater area than other portions of the body of the housing 3. Thus, thinning the flat surface portion 3d as much as possible while maintaining the strength can prevent an increase in weight.

On the other hand, as illustrated in FIG. 3, the average thickness of the side surface portion 3b may be greater than the average thickness of the slope portion 3c in the housing 3. Further, the thickness of the housing 3 is varied in descending order, that is, in order of the thickness (t_b) of the thickest portion of the side surface portion 3b, the thickness (t_c) of the thickest portion of the slope portion 3c, and the thickness (t_d) of the thickest portion of the flat surface portion 3d. The side surface portion 3b of the housing 3 is particularly likely to receive an impact due to falling during transportation or installation, but this structure can reduce an external impact. The thickness of each portion is appropriately selected to maintain the load capacity and the operability. For example, the thickness t_b is selected from the range of 1.5 mm to 10 mm, the thickness t_c is selected from the range of 0.8 mm to 2.0 mm, and the thickness t_d is selected from the range of 0.5 mm to 1.5 mm. The slope portion 3c in the housing 3 does not have to be provided on four sides. The slope portion 3c may be provided on only two opposing sides or may be provided at least on one side. In FIG. 3, the thickness has been described using the thickness of the thickest portion of each portion of the housing 3, but determination of the thickness is not limited to this. For example, the thickness may be varied in descending order, that is, in order of the average thickness of the side surface portion 3b, the average thickness of the slope portion 3c, and the average thickness of the flat surface portion 3d. In this manner, increasing the thickness of portions in accordance with the likelihood of external impacts being exerted on the portions can enhance the strength of the housing while the operability (portability) of the housing is maintained.

As in the above-described structure, the housing of the radiographic apparatus has a slope portion and the thickness of at least part of the slope portion is greater than the thickness of the thickest portion of the flat surface portion. The radiographic apparatus having this structure can reduce stress concentration that occurs at or around the slope portion upon receipt of an external force. Furthermore, the radiographic apparatus having this structure can prevent bending around the slope portion or buckling of the slope portion. In addition, the radiographic apparatus can maintain the operability when the radiographic apparatus is inserted into a lower portion of a test subject during radiographing. Thus, the radiographic apparatus can have a high operability and maintain the strength of the housing.

### Second Embodiment

Referring to FIG. 4, a second embodiment is described. The second embodiment is different from the first embodiment in the structure of the slope portion of the housing. The second embodiment is described in detail below.

As illustrated in FIG. 4, as in the case of the first embodiment, the housing according to the second embodiment has a thickness such that the average thicknesses of the side surface portion 3b and the slope portion 3c are greater than the average thickness of the flat surface portion 3d.

The average thickness of a portion of the housing 3 extending outward beyond an orthographic projection area, obtained by orthographically projecting the radiation sensor panel 1 toward the flat surface portion 3d, is greater than the average thickness of the orthographic projection area.

This structure can increase the capacity of the housing 3. Moreover, this structure can increase the distance between the inner wall of the housing 3 and the enclosure, such as the radiation sensor panel 1, the flexible circuit board 4, and the control boards 5. This structure can thus minimize the likelihood of the housing 3 coming into contact with the enclosure as a result of the housing 3 being bent due to, for example, an external load on the housing 3.

This structure can prevent an increase in weight and a reduction of the exterior capacity of the radiographic apparatus while the slope portion is provided to improve the operability of the radiographic apparatus.

### Third Embodiment

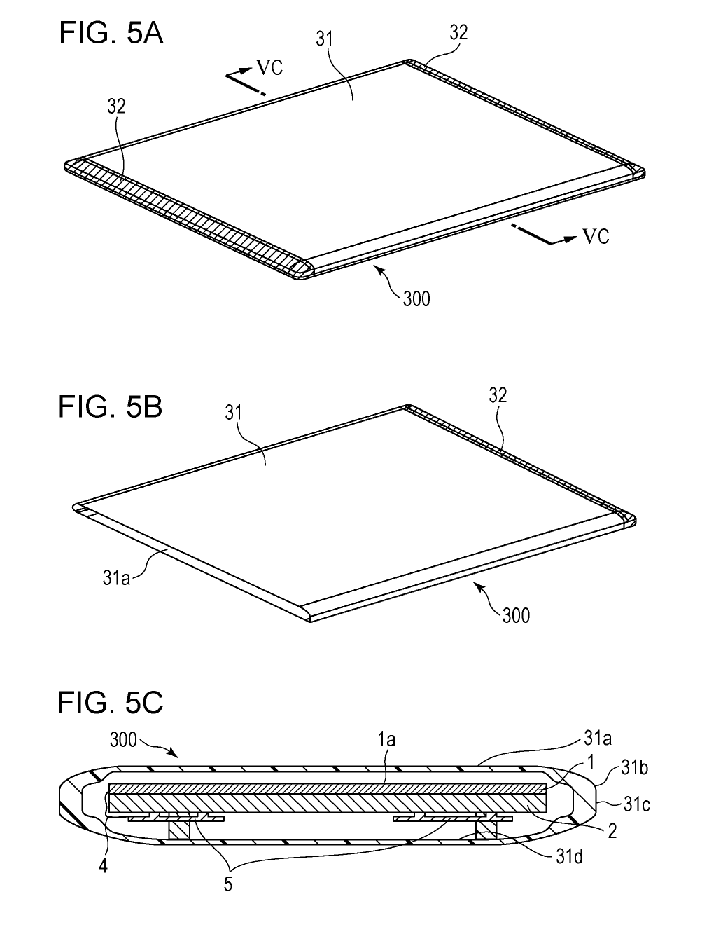

Referring to FIGS. 5A to 5C, a third embodiment is described. FIG. 5A is a perspective view of a radiographic apparatus according to a third embodiment. FIG. 5B is a perspective view of the radiographic apparatus according to the third embodiment in the state where lid members are removed. FIG. 5C is a cross-sectional view of the radiographic apparatus taken along the line VC-VC in FIG. 5A. Unlike the other embodiments, the housing according to this embodiment has a structure in which two opposing side portions of the side surface portion, the slope portion, and the flat surface portion are integrated into one unit. The structure of the third embodiment is described in detail below.

A housing 31 has an incident portion 31a, a side surface portion 31b, a slope portion 31c, and a flat surface portion 31d. The housing 31 is made of a carbon fiber reinforced plastic (CFRP). The housing 31 having this structure has a high radiation permeability to allow radiation to penetrate therethrough, is light in weight, and has a predetermined strength against impacts. As illustrated in FIG. 5B, the housing 31 is shaped in a hollow tube. Thus, the housing 31 is likely to have a mechanical strength, including a distortion resistance, higher than the housing according to the first embodiment. Moreover, as illustrated in FIGS. 5A to 5C, the housing 31 has openings 31e on two opposing sides. This structure allows the radiation sensor panel 1 to be inserted into the housing 31 through the openings 31e and thus facilitates an assembly of a radiographic apparatus 300. The housing 31 includes lid members 32 to form side walls and cover the openings 31e. The lid members 32 are made of aluminum, which is a metal. The lid members 32 may be covered with, for example, protection covers. The protection covers made of a material softer than metal such as resin can improve the operability of the housing 31. Installing the lid members 32 allows the housing 31 to form a closed space. In addition, the lid members 32 can prevent a reduction of the mechanical strength around the openings 31a.

As described above, the housing has a structure in which two opposing side portions of the side surface portion, the slope portion, and the flat surface portion are integrated into one unit. This structure can enhance the mechanical strength while the slope portion is provided in the radiographic apparatus for operability improvement. This structure can prevent an increase in weight and reduce an impact force exerted on the housing.

Throughout the first embodiment to the third embodiment, the case where the radiographic apparatus has a housing having an incident surface located adjacent to the detection surface 1a has been described. However, the present invention is not limited to this case. The housing may include an incident portion, which allows radiation to penetrate therethrough and which is located on the side of the radiation sensor panel 1 opposite to the detection surface 1a, a slope portion, which is located adjacent to the detection surface 1a and inclined with respect to the thickness direction of the housing, and a flat surface portion, which is located adjacent to the detection surface la and extends substantially parallel to the flat portion of the incident portion. In this case, the fluorescent substance emits light at a position close to the photoelectric converting elements, which are converting elements. Thus, the intensity of detectable light can be enhanced and scattering of light can be minimized.

In addition, the structure of the housing is not limited to those according to the embodiments. For example, the incident portion and the side surface portion may be integrated into one unit.

### Fourth Embodiment

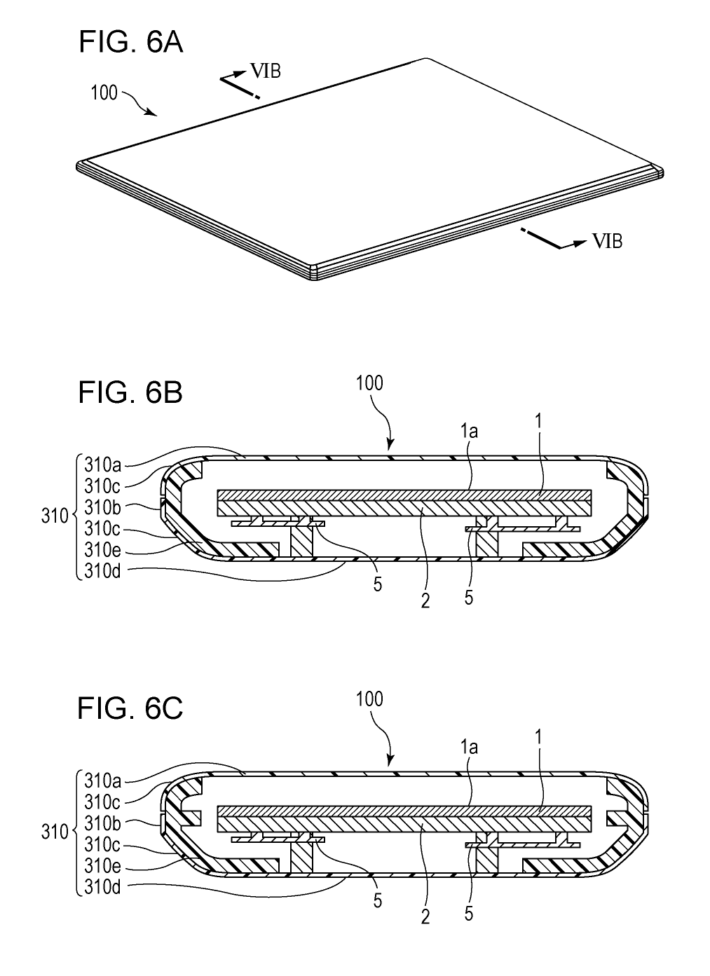

Referring to FIGS. 6A to 6C, a fourth embodiment is described. FIG. 6A is a perspective view of a radiographic apparatus according to a fourth embodiment. FIG. 6B is a cross-sectional view of the radiographic apparatus taken along the line VIB-VIB in FIG. 6A. The radiographic apparatus according to the fourth embodiment is different from those according to the other embodiments in that the radiographic apparatus according to the fourth embodiment additionally includes a side structural member 310e. Thus, the average thickness of the slope portions can be regarded as a sum of the thickness of a slope member of the side surface portion of the housing and the thickness of a structural member (side structural member 310e).

A housing 310 encloses the radiation sensor panel 1 as in the case of the housing according to another embodiment. In the fourth embodiment, as illustrated in FIG. 6B, the housing 310 includes an incident portion (incident member) 310a, a side surface portion (side surface member) 310b, a slope portion (slope member) 310c, a flat surface portion (flat surface member) 310d, and a side structural member 310e. The side structural member 310e is disposed on at least the inner side of the slope portion 310c. In this embodiment, for example, the side structural member 310e is disposed on the housing 310 over an area extending between the incident portion 310a, the side surface portion 310b, the slope portion 310c, and the flat surface portion 310d. Here, the side structural member 310e is separable from at least one of the incident portion 310a and the body (portion of the housing 310 excluding the incident portion 310a). The incident portion 310a is located adjacent to the detection surface 1a of the radiation sensor panel 1. The incident portion 310a has a flat portion that allows radiation to penetrate therethrough. Thus, it is desirable that the radiation permeability at which radiation is allowed to pass from the flat portion of the incident portion 310a to the detection surface 1a be higher than the radiation permeability at which radiation is allowed to pass from the flat surface portion 310d to the detection surface 1a.

Use of a material that hinders a continuous change of the thickness between the incident portion 310a and the body may hamper forming the structure according to any of the first to third embodiments. Examples of the materials of the incident portion 310a and the body include metal plates and fiberglass reinforced plastic (FRP) sheets such as prepreg. Thus, in the fourth embodiment, the use of the side structural member 310e allows the incident portion 310a and the body to have any of a variety of shapes. In other words, in the radiographic apparatus according to the embodiment, the strength of the housing 310 can be enhanced using the side structural member 310e while the incident portion 310a and the body maintain their operability. Here, examples of the material of the side structural member 310e include resin and fiber-reinforced resin. In this case, the side structural member 310e can be formed by a selective, highly formative method. As in the other embodiments, the side structural member 310e can be integrated with other components of the housing 310 and the thickness of the housing can be changed with there being the side surface portion 310b, the slope portion 310c, and the flat surface portion 310d. Thus, the housing 310 enables minimization of stress concentration that can occur at or around the slope portion upon receipt of an external force and the occurrence of buckling of the slope portion. As illustrated in FIG. 6B, the side structural member 310e has a function of combining the incident portion 310a and the body (side surface portion 310b, slope portion 310c, and flat surface portion 310d) together.

Here, the side structural member 310e is made of a material such as resin or fiber-reinforced resin. Moreover, the side structural member 310e may be inseparably integrated with either the incident portion 310a or the body. The shape of the side structural member 310e is not limited to the one illustrated in FIG. 6B. For example, as illustrated in FIG. 6C, the side surface portion 310b may be modified from a shape having a uniform thickness to a rib shape, in which the thickness is varied. This structure is stronger against deformation that would occur due to an external force. In this modification, the thickness of the side structural member 310e may be varied in the manner as illustrated in FIG. 3 in descending order, that is, in order of the thickness (t_b) of the thickest portion of the side surface portion 310b, the thickness (t_c) of the thickest portion of the slope portion 310c, and the thickness (t_d) of the thickest portion of the flat surface portion 310d. This structure can reduce an external impact resulting from falling during transportation or installation.

In the manner as described above, disposing the structural member on the inner side of the housing enables securing the operability and the strength of the radiographic apparatus.

### APPLICATION EXAMPLE

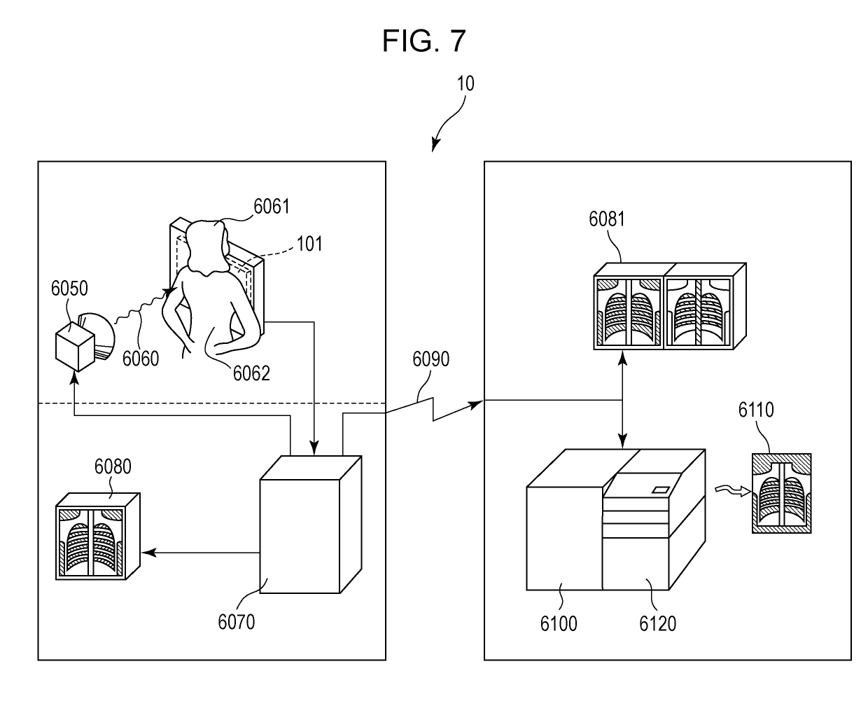

FIG. 7 illustrates an example in which the radiographic apparatus according to any of the first to fourth embodiments is used in a radiographic system 10. A radiographic apparatus 101 according to any of the embodiments of the invention is used in the radiographic system 10.

The radiographic system 10 includes an X-ray tube 6050 serving as a radiation source, a radiographic apparatus 101, an image processor 6070 serving as a signal processor, and displays 6080 and 6081 serving as displaying devices. The radiographic system 10 also includes a film processor 6100 and a laser printer 6120.

Radiation (X-rays) 6060 generated by the X-ray tube 6050 serving as a radiation source penetrates through a radiograph portion 6062 of a test subject 6061 and enters the radiographic apparatus 101. The radiation that has entered the radiographic apparatus 101 contains information of the inside of the radiograph portion 6062 of the test subject 6061.

When receiving radiation, the radiographic apparatus 101 obtains electric information of the radiograph portion 6062 of the test subject 6061. This information is converted into a digital form and then output to the image processor 6070 serving as a signal processor.

A computer including a CPU, a RAM, and a ROM is used as an example of the image processor 6070 serving as a signal processor. The image processor 6070 also includes a recording medium that can record various information and serves as a recording device. For example, the image processor 6070 includes, as recording devices, a HDD, a SSD, and a recordable optical disk drive. Alternatively, the image processor 6070 may be connected with external recording devices such as a HDD, a SSD, and a recordable optical disk drive.

The image processor 6070 serving as a signal processor performs predetermined signal processing on this information and causes the displays 6080, serving as displaying devices, to display the processed information thereon. Thus, the test subject or a technician can observe the image. The image processor 6070 can thus record this information on the HDD, the SSD, and the recordable optical disk drive, serving as recording devices.

The image processor 6070 may include an interface that can transmit information to the outside and serves as an information transmitting device. Examples of such an interface serving as an information transmitting device include an interface that is connectable with a LAN or a telephone line 6090.

The image processor 6070 can transmit this information to a remote place through the interface serving as a transmitting device. For example, the image processor 6070 transmits this information to a doctor room located away from a X-ray room in which the radiographic apparatus 101 is located. Thus, a doctor or the like can diagnose the test subject at a remote place. The radiographic system 10 can record this information on a film 6110 using a film processor 6100 serving as a recording device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-125731 filed Jun. 18, 2014 and No. 2015-061684 filed Mar. 24, 2015, which are hereby incorporated by reference herein in their entirety.

What is claimed is:

1. A radiographic apparatus comprising:
a radiation sensor panel having a detection surface on which a converting element that detects radiation or light is disposed; and
a housing that encloses the radiation sensor panel,
wherein the housing includes
an incident portion through which the radiation enters the radiographic apparatus, wherein the incident portion is located adjacent to the detection surface of the radiation sensor panel,
a slope portion that is located at an end portion of the housing and on a side of the radiation sensor panel opposite to the detection surface, wherein the slope portion is inclined with respect to a direction of a thickness of the housing, and
a flat surface portion that is located on the side of the radiation sensor panel opposite to the detection surface and that is substantially parallel to a flat portion of the incident portion, and
wherein the slope portion has an average thickness that is greater than an average thickness of the flat surface portion.

2. The radiographic apparatus according to claim 1, wherein the housing has a thickness such that a difference in thickness between the slope portion and the flat surface portion gradually decreases.

3. The radiographic apparatus according to claim 1,
wherein the housing also includes a side surface portion located at an outer edge of the radiation sensor panel, and
wherein the side surface portion has an average thickness that is greater than an average thickness of the flat surface portion.

4. The radiographic apparatus according to claim 3, wherein the housing has a thickness such that at least part of the side surface portion has a thickness greater than the average thickness of the slope portion.

5. The radiographic apparatus according to claim 3, wherein the housing has a thickness such that the average thickness of the side surface portion is greater than the average thickness of the slope portion.

6. The radiographic apparatus according to claim 3, wherein the housing has such a structure that the slope portion, the side surface portion, and the flat surface portion are integrated into one unit.

7. The radiographic apparatus according to claim 6,
wherein the housing has such a structure in which the incident portion, the slope portion, the side surface portion, and the flat surface portion are integrated into one unit, and
wherein the side surface portion has an opening on at least one side of the side surface portion.

8. The radiographic apparatus according to claim 1, wherein the housing has a thickness such that at least part of the slope portion has a thickness that is the same as the average thickness of the flat surface portion.

9. The radiographic apparatus according to claim 1, wherein the housing has a thickness such that an average thickness of a portion of the housing extending outward beyond an orthographic projection area, obtained by orthographically projecting the radiation sensor panel toward the flat surface portion, is greater than an average thickness of the orthographic projection area.

10. The radiographic apparatus according to claim 1, wherein the housing has the slope portion on each of opposing two sides.

11. The radiographic apparatus according to claim 1,
wherein the housing also includes a structural member, and
wherein the average thickness of the slope portion is a sum of a thickness of a slope member of a side surface portion of the housing and a thickness of the structural member.

12. The radiographic apparatus according to claim 11, wherein the structural member is disposed to extend over the incident portion, the slope portion, and the flat surface portion.

13. The radiographic apparatus according to claim 11, wherein the structural member is coupled with the incident portion and the slope portion.

14. A radiographic apparatus, comprising:
a radiation sensor panel having a detection surface on which a converting element that detects radiation or light is disposed; and
a housing that encloses the radiation sensor panel,
wherein the housing includes
an incident portion through which the radiation enters the radiographic apparatus, wherein the incident portion is located on a side of the radiation sensor panel opposite to the detection surface,
a slope portion that is located adjacent to the detection surface, wherein the slope portion is inclined with respect to a direction of a thickness of the housing, and
a flat surface portion that is located adjacent to the detection surface and that is substantially parallel to the incident portion, and
wherein the slope portion has an average thickness that is greater than an average thickness of the flat surface portion.

15. The radiographic apparatus according to claim 14,
wherein the housing also includes a structural member, and
wherein the average thickness of the slope portion is a sum of a thickness of a slope member of a side surface portion of the housing and a thickness of the structural member.

16. A radiographic system, comprising:
the radiographic apparatus according to claim 1; and
a signal processor that processes signals from the radiographic apparatus.

17. A radiographic system, comprising:
the radiographic apparatus according to claim 14; and
a signal processor that processes signals from the radiographic apparatus.
